(19) United States
(12) Patent Application Publication
(10) Pub. No.: US 2026/0295229 A1
(43) Pub. Date: Oct. 1, 2026

(54) SYSTEM AND METHOD FOR DELIVERY OF GAS TO A TISSUE

(71) Applicant: BEYOND AIR, INC., Garden City, NY (US)

(72) Inventors: Frederick John MONTGOMERY, Sun Prairie, WI (US); Ryan MAGEE, Garden City, NY (US); Steven A. LISI, Rockville Centre, NY (US)

(21) Appl. No.: 19/393,717

(22) Filed: Nov. 19, 2025

Related U.S. Application Data
(63) Continuation of application No. PCT/US2024/029884, filed on May 17, 2024.
(60) Provisional application No. 63/467,625, filed on May 19, 2023.

(51) Int. Cl. A61M 37/00

(52) U.S. Cl. CPC A61M 37/00; A61M 2202/0007; A61M 2202/0275; A61M 2205/3334; A61M 2205/7536

(57) ABSTRACT

Embodiments of the present invention provide for systems and methods of delivering medical gas to a tissue. The exemplary systems can be operated in “closed circuit” or “open circuit” system configurations. In embodiments, the “closed circuit” system comprises: a housing containing: a medical gas container including the medical gas, a medical gas delivery circuit, a medical gas extraction circuit, and a safety absorption module; a medical gas delivery module; and a medical gas extraction module. In embodiments, the “open circuit” system comprises: a housing containing: a medical gas container including the medical gas, a medical gas delivery circuit, a safety absorption module; a medical gas delivery module, and, optionally, a medical gas extraction circuit.

### RELATED APPLICATIONS

This application is continuation of International Application No. PCT/US2024/029884, which designated the United States and was filed on May 17, 2024, published in English, which claims the benefit of U.S. Provisional Application No. 63/467,625 filed May 19, 2023. The entire teachings of the above applications are incorporated herein by reference.

### FIELD

The present invention, in some embodiments thereof, relates to medical equipment and, more particularly, to a system and method for delivery of gas, e.g., gaseous nitric oxide (NO) to a tissue, preferably a cancerous tissue.

### BACKGROUND OF THE INVENTION

In 2018, about 18.1 million new cases of cancer and 9.6 million deaths from cancer have been reported worldwide (Ferlay, J. et al. Estimating the global cancer incidence and mortality in 2018: GLOBOCAN sources and methods. Int. J. cancer 144, 1941-1953 2019). Only in the US, there are estimated 1,762,450 cancer cases and 606,880 cancer deaths since January 2019 (Siegel, R. L., Miller, K. D. & Jemal, A. Cancer statistics, 2019. CA. Cancer J. Clin. 69, 7-34).

Cancer has traditionally been treated with surgery, cytotoxic chemotherapy, and/or radiotherapy. The past decades have seen the emergence of immunomodulating drugs that use the power of the body's own immune response to prevent, control, and eliminate tumors. Despite the promise of these immunotherapies, they are effective only in a select group of cancers and usually in a minority of patients with those cancers. In addition, resistance develops to many cancer treatments that target single molecular mutations or cancer pathways, so they have only modestly affected survival in some cancers.

While immunomodulating therapies have shown promise in improving outcomes for hematological cancers, significant work remains in treating solid tumors. Solid tumors represent approximately 90% of adult human cancers. In addition, metastatic disease is responsible for 90% of deaths from solid tumors.

Nitric oxide (NO) is a short-lived, endogenously produced gas that acts as a signaling molecule in the body. Increasing evidence highlights its wide spectrum of action in different pathologic conditions, including cancer and involvement in immune cell signaling against pathogens.

Preclinical studies testing the effect of exogenously administered nitric oxide (NO) demonstrated its anti-cancer properties and suggested that NO may serve as a potent tumoricidal ablation agent. While NO at low doses may possess pro-oncogenic properties; at high doses, NO may have a role in cancer therapy either as a single agent or in combination with other antineoplastic compounds. More specifically, high doses of NO were shown to promote oxidative/nitrosative stress and DNA damage.

### SUMMARY OF THE INVENTION

The systems of the present embodiments can effectively administer medical gases, such as gaseous nitric oxide (gNO) to treat tumors. Compared to conventional techniques, the systems of the present embodiments are more targeted in treating tumors, particularly cancerous cells in vivo.

The systems of the present embodiments can effectively deliver gNO in treatment regimes, and can deliver gNO to target sites, with minimal damage, and preferably without damaging, healthy adjacent host cells.

Embodiments of the present invention provide for systems and methods of delivering medical gas to a tissue. The exemplary systems can be operated in “closed circuit” or “open circuit” system configurations.

In embodiments, the “closed circuit” system comprises: a housing containing: a medical gas container including the medical gas, a medical gas delivery circuit, a medical gas extraction circuit, and a safety absorption module; a medical gas delivery module; and a medical gas extraction module, wherein the medical gas delivery module is attached to the medical gas delivery circuit to allow the medical gas to be delivered to the tissue and the medical gas extraction module is in communication with the medical gas delivery module to return excess medical gas to the medical gas extraction circuit. In this regard, each of the medical gas delivery circuit and the medical gas extraction circuit independently comprises electromechanical components that modulate the pressure and flow rate of the medical gas from the medical gas container to the target tissue (e.g., Attorney tumor) and back to the system, creating the “closed circuit” system. In embodiments, the medical gas delivery circuit and/or the medical gas extraction circuit further comprise safety control systems.

In embodiments, the “open circuit” system comprises: a housing containing: a medical gas container including the medical gas, a medical gas delivery circuit, a safety absorption module; a medical gas delivery module, and, optionally, a medical gas extraction circuit, wherein the medical gas delivery module is attached to the medical gas delivery circuit to allow the medical gas to be delivered to the tissue. In this regard, the medical gas delivery circuit comprises electromechanical components that modulate the pressure and flow rate of the medical gas from the medical gas container to the target tissue (e.g., tumor), creating the “open circuit” system. In embodiments, the medical gas delivery circuit further comprises safety control systems.

Unlike the “closed circuit” system configuration, in the “open circuit” system configuration, there is no medical gas extraction module and, therefore, no medical gas extraction tubing in communication with the delivery applicator to return medical gas to the system, in particular, to the medical gas extraction circuit, if present.

In embodiments, the medical gas is gaseous nitric oxide (gNO). In embodiments, the gaseous nitric oxide is ultra-high concentration nitric oxide (UNO).

Although the terms gNO and UNO refer to the same medical gas and can be used interchangeably, UNO is preferably defined as the delivery of gaseous nitric oxide in a preferably inert gas such as N₂ at a concentration of between about 10,000 and 1,000,000 ppm, as is described in further detail hereinafter. Any reference to gNO having a concentration of 10,000 ppm or higher is also intended to refer to UNO.

The medical gas delivery module and the medical gas extraction module respectively comprise medical gas delivery tubing, including a delivery applicator, and medical gas extraction tubing, wherein the medical gas delivery tubing is attached to the medical gas delivery circuit to allow the medical gas (e.g., ultra-high concentration gaseous nitric oxide (UNO)) to be delivered to the tissue through the delivery applicator and the medical gas extraction tubing is in communication with the delivery applicator to return medical gas to the medical gas extraction circuit. The safety absorption module is in communication with at least one of (i) the medical gas delivery circuit in order to neutralize any unwanted leaks of the medical gas during administration and (ii) the medical gas extraction circuit in order to neutralize any excess medical gas extracted from the tissue.

In an embodiment, the distal end of the extraction tubing is in communication with the distal end of the delivery applicator, where the communication between the delivery applicator and the extraction tubing is airtight. As described further herein, the communication between the delivery applicator and the extraction tubing creates a closed system, wherein the medical gas is delivered through the medical gas delivery circuit to the delivery tubing and the delivery applicator, wherein the medical gas is delivered to the tissue, and wherein the medical gas that does not exit the delivery applicator (is not delivered to the tissue), referred to herein as excess gas, flows into the extraction tubing and into the medical gas extraction circuit and finally to the safety absorption module where any toxic gas is captured before venting the cleaned excess gas into the ambient air. In this manner, no excess medical gas is vented into the ambient air.

In embodiments, the medical gas delivery tubing and the medical gas extraction tubing are single-use only and disposed of after each treatment.

The delivery applicator is designed for the delivery of UNO into the target tissue. In embodiments, the delivery applicator is single-use only and disposed of after each treatment.

In embodiments, the safety absorption module is single-use only and disposed of after each treatment.

The housing securely holds the system components for efficient and safe administration of the medical gas, e.g., UNO. In embodiments, the housing can be individualized to each patient's treatment by the concentration of the medical gas, the delivery and/or extraction tubing, and the delivery applicator provided. In embodiments, the housing allows for ease of use, repacking upon system return, and a safety mechanism for handling excess UNO and risk controls during shipping, including cylinder control to avoid user handling/shipping concerns.

In embodiments, the medical gas is in a container. In embodiments, the medical gas container comprises an outlet regulator, cylinder and associated valves mounted thereon and connected to the medical gas delivery circuit.

In embodiments, the medical gas container is packaged within the housing. By packaging the medical gas container in the housing, shipping risks associated with shipping medical gases, as is especially important if that gas is toxic, may be avoided. The housing is airtight and provides the exhaust of the system through a gas filter, removing risk of toxic gas escaping into ambient air.

In embodiments, the medical gas container is reusable. This is particularly advantageous as the container can be closed as to not allow evacuation into room air after treatment and shipped, separately or within the housing, back to the point of manufacturer for refill or disposal.

In embodiments, the volume of medical gas container is sufficiently small so that the amount of medical gas in medical gas container is not more than the typical gas dose to be delivered to the tissue for treatment. This can be particularly advantageous when the gas is toxic, as in the case of gaseous nitric oxide (gNO) at levels above 22,000ppm, because in the event of undesired leakage of the gas, the total amount of gas that can be leaked is small, thereby reducing the risk of inhaling a hazardous concentration of the gas by the subject or medical personnel.

In embodiments, the medical gas container comprises from about 1,000 ppm to 1,000,000 ppm of the gas, or any intermediate subrange therebetween. In embodiments, the medical gas container comprises from about 1,000 ppm to about 200,000 ppm, or from about 1,000 ppm to about 100,000 ppm, preferably from about 10,000 ppm to about 500,000 ppm, or from about 10,000 ppm to about 200,000 ppm, or from about 10,000 ppm to about 100,000 ppm, or from about 20,000 ppm to about 100,000 ppm, or from about 25,000 ppm to about 100,000 ppm, or from about 25,000 ppm to about 75,000 ppm, or from about 10,000 ppm to about 50,000 ppm, or from about 50,000 ppm to about 100,000 ppm, including any intermediate values and sub-ranges between any of the foregoing, or is about 50,000 ppm. In embodiments, the medical gas container comprises 10,000 ppm of the gas. In embodiments, the medical gas container comprises 20,000 ppm of the gas. In embodiments, the medical gas container comprises 25,000 ppm of the gas. In embodiments, the medical gas container comprises 50,000 ppm of the gas. In embodiments, the medical gas container comprises 100,000 ppm of the gas. In embodiments, the medical gas container comprises 150,000 ppm of the gas. In embodiments, the medical gas container comprises 200,000 ppm of the gas. In embodiments, the medical gas container comprises 250,000 ppm of the gas.

The gas pressure in the container is preferably enough to drive the delivery system to avoid repressurising the gas, e.g., less than 30 bar, with a delivery from about 1 bar to about 5 bar. In embodiments, the medical gas container comprises 50 bars of the gas. In embodiments, the medical gas container comprises 150 bars of the gas.

In embodiments, the volume of the medical gas container is between 10 standard microliters and 1000 standard liters.

In an embodiment, the medical gas delivery circuit comprises one or more valves, one or more regulators, one or more cylinder manifolds, and one or more flow sensors. The medical gas delivery circuit provides fluid communication between the medical gas container and the delivery tubing and applicator, wherein the medical gas flows from the medical gas container through the medical gas delivery circuit, out the gas outlet, through the delivery tubing to the delivery applicator to the target tissue.

In embodiments, the medical gas delivery circuit comprises a gas manifold, a pressure regulator, a first valve, a flow sensor, a gas filter, and a second valve, wherein the flow sensor is arranged after the first and second valves. In embodiments, the second valve is a three-port valve having a first port for receiving the medical gas from the medical gas container, a second port for receiving a purging gas, e.g., ambient air, and a third port in communication with the delivery tubing. The three-port valve being switchable between a first state at which the first port fluidly connects to the third port, a second state at which the second port fluidly connects to the third port, and a third state at which the valve is closed. In embodiments, the first and/or second valve can independently be of any type, such as, but not limited to, a ball valve, a gate valve, a plunger valve, a solenoid, a butterfly valve or the like.

In embodiments, the purging gas is ambient air. In embodiments, purging gas is nitrogen or argon.

According to some embodiments of the invention, the medical gas delivery circuit further comprises one of a flow metering and a flow control system.

In embodiments, the flow metering system can be one of a mass flow meter, rotameter, etc. In embodiments, the flow control system can be at least one of a flow limiter and a flow controller. The flow controller can be one of a digital or analog mass flow controller.

In embodiments, the flow control system can comprise an orifice and a valve. According to some embodiments of the invention, the valve of the flow control system is a solenoid valve.

In embodiments, the medical gas delivery circuit is in communication with the delivery tubing. The connection of the gas delivery circuit and delivery tubing to the respective ports is of a fast connection type.

In embodiments, the applicator can be of any type that has an outlet through which a flow of gas can exit. Typically, but not necessarily, the applicator is a transcutaneous device, e.g., a perforated catheter or needle dependent on the physiological area or treatment of the respective tumor.

In embodiments, the extraction tubing has a member or diaphragm to attach to the distal end of the delivery applicator thereby making an airtight connection. The connection is a risk control to prevent the medical gas from being released into ambient air.

In embodiments, the extraction tubing is of a larger volume than the delivery tubing. The extraction tubing has a check valve in order to draw in ambient air thereby diluting the medical gas to non-toxic levels.

In embodiments, the medical gas extraction circuit comprises a connection port for the extraction tubing, gas sensor, pump, and a flow sensor. In embodiments, the connection port is a valve. In embodiments, the connection port is of a fast connection type. In some embodiments, the extraction circuit may comprise one or more biological filters, which are configured to protect the extraction circuit, the delivery device, and/or the patient from cross contamination.

In embodiments, the medical gas sensor is configured to determine if the medical gas is flowing into the medical gas extraction circuit. In embodiments, the medical gas pump is configured to operate at a sufficient flow as to dilute the extracted excess medical gas to below ambient concentrations. In embodiments, the flow sensor is configured to assure there is a flow of medical gas in the medical gas extraction circuit.

In embodiments, the excess medical gas flows through the medical gas extraction circuit and to a safety absorption module. The safety absorption module is used to filter the excess medical gas to remove any toxic components from the medical gas. After the gas passes through the safety absorption module, it is exhausted from the system into ambient air. In embodiments, the safety absorption module is removable and exchangeable in between treatments. In embodiments, the safety absorption module is reusable. In embodiments, the safety absorption module is disposed of after one use, i.e., one treatment.

In embodiments, the system may further comprise a pressure sensor to measure the intra-tumoral pressure of the tumor being treated. In embodiments, the pressure sensor can measure a pressure in a range 0.001 Bar to 5 Bar. In embodiments, the pressure sensor can be implemented in the medical gas delivery circuit. For example, the pressure sensor can be implemented in the flow controller. In other embodiments, the pressure sensor can be implemented in the delivery tubing, the applicator, and/or the extraction tubing. In embodiments, the pressure sensor can be used to discontinue flow, or intermittently stop the flow, of the medical gas at the injection site to disperse pressure under the skin or actively remove the pressure using, for example, the medical gas extraction circuit.

In embodiments, all of the components of the system are monitored and controlled in their various exemplary embodiments of the invention and the fluid communications between constituent components are electronically connected with the use of an electronic controller. The electronic controller may consist of but is not limited to: a printed circuit board, microcontroller(s) or programmable ladder logic controller(s). In embodiments, the system may comprise more than one electronic controller.

In some embodiments, the medical gas extraction circuit is single-use only and disposed of after each treatment.

According to some embodiments of the invention, the delivery tubing, the applicator, and the extraction tubing are disposable.

According to some embodiments of the invention, the medical gas delivery circuit further comprises a flow control system. The flow control system comprises an orifice and a valve.

According to some embodiments of the invention, the valve of the flow control system is a solenoid valve.

According to some embodiments of the invention, the flow control system comprises a flow controller and a flow limiter for limiting flow rate of the gas before entering the flow controller.

According to some embodiments of the invention, the system comprises an adjustable pressure regulator, in fluid communication with the third port of the valve, and being configured for maintaining a pressure which is below a predetermined threshold when the valve assumes the first state, and a pressure which is above the predetermined threshold when the valve assumes the second state.

According to some embodiments of the invention, the electronic controller is configured to control the three-port valve to perform purging in multiple cycles.

According to an aspect of some embodiments of the present invention, there is provided a method for delivery of medical gas to a tissue, the method comprises: in the system as delineated above and optionally and preferably as further detailed below: connecting the medical gas container to the medical gas delivery circuit; switching the valve to the first state, thereby delivering the medical gas to the tissue and back to the system through the medical gas extraction circuit; switching the valve to the second state to draw in a purging gas and delivering the purging gas through the applicator and the extraction circuit, thereby purging the system.

According to embodiments of the invention, the medical gas is gaseous nitric oxide (gNO). In embodiments, the medical gas is ultra-high concentration nitric oxide (UNO). According to some embodiments of the invention, the subject is a mammal.

According to some embodiments of the invention, the subject is a human.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic illustration of a closed circuit system for delivery of gas to a tissue of a subject, according to one embodiment of the present invention;

FIG. 1B is a schematic illustration of an open circuit system for delivery of gas to a tissue of a subject, according to one embodiment of the present invention;

FIG. 1C is a schematic illustration of an open circuit system for delivery of gas to a tissue of a subject, according to another embodiment of the present invention;

FIG. 2A is a flowchart diagram illustrating a method suitable for delivery of a gas to a tissue by a closed circuit system, according to some embodiments of the present invention; and

FIG. 2B is a flowchart diagram illustrating a method suitable for delivery of a gas to a tissue by an open circuit system, according to some embodiments of the present invention.

### DESCRIPTION OF SPECIFIC EMBODIMENTS OF THE INVENTION

The present invention, in some embodiments thereof, relates to medical equipment and, more particularly, to a system and method for delivery of gas, e.g., gaseous nitric oxide (NO) to a tissue. Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

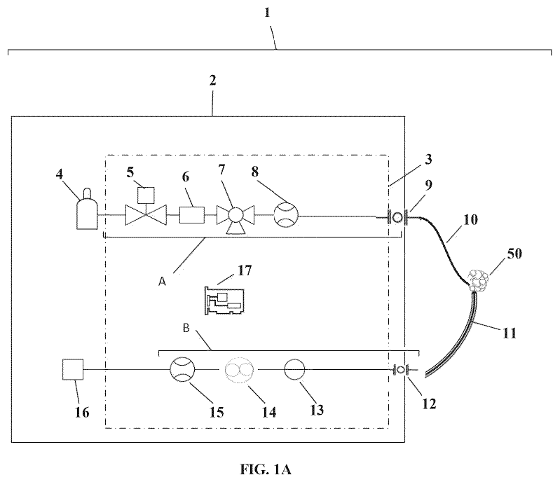

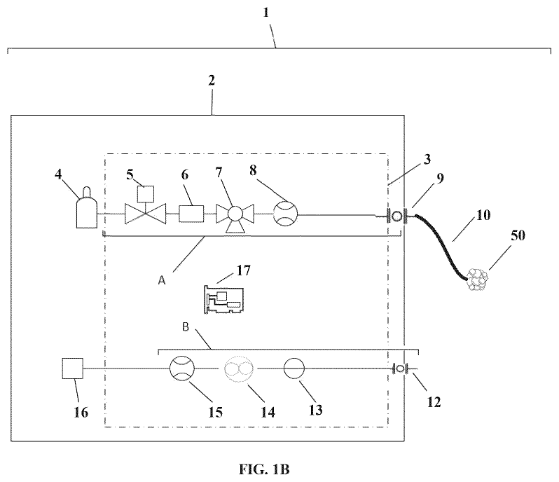

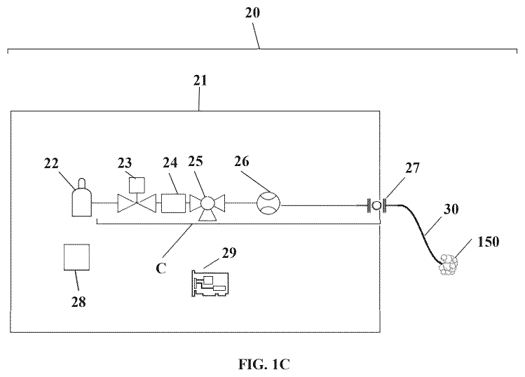

Referring now to the drawings, FIG. 1A illustrates a system 1 for delivery of medical gas to a tissue 50 of a subject, according to some embodiments of the present invention. The subject is optionally and preferably a mammalian subject, more preferably a human subject. The tissue 50 is typically a tumor or a metastasis, and the gas is delivered for the purpose of treating the tumor or metastasis. In some embodiments of the present invention, the tumor is a malignant tumor.

Tumors treatable by system 1 include, but are not limited to, breast tumor, brain tumor, neuroblastoma, thyroid gland tumor, gestational trophoblastic tumor, uterine sarcoma, carcinoid tumor, colon carcinoma, esophageal carcinoma, hepatocellular carcinoma, liver carcinoma, lymphoma, plasma cell neoplasm, mesothelioma, thymoma, alveolar soft-part sarcoma, angiosarcoma, epithelioid sarcoma, extraskeletal chondrosarcoma, fibrosarcoma, leiomyosarcoma, liposarcoma, malignant fibrous histiocytoma, malignant hemangiopericytoma, malignant mesenchymoma, malignant schwannoma, synovial sarcoma, melanoma, neuroepithelioma, osteosarcoma, leiomyosarcoma, Ewing sarcoma, osteosarcoma, rhabdomyo-sarcoma, hemangiocytoma, myxosarcoma, mesothelioma (e.g., lung mesothelioma), granulosa cell tumor, thecoma cell tumor and Sertoli-Leydig tumor.

System 1 can also be used to treat cancers, such as, but not limited to, vaginal cancer, vulvar cancer, cervical cancer, endometrial cancer, ovarian cancer, rectal cancer, salivary gland cancer, laryngeal cancer, nasopharyngeal cancer, many lung metastases and acute or chronic leukemia (e.g., lymphocytic, Myeloid, hairy cell).

The medical gas is optionally and preferably gNO or UNO, but other medical gases are also contemplated, e.g., nitrogen (N₂), nitrous oxide (N₂O), carbon dioxide (CO₂), carbon monoxide (CO), helium (He), sulfur hexafloride (SF₆), perfluoropropane (C₃F₈), perfluororoethane (C₂F₆).

System 1 comprises a housing 2, medical gas delivery module 10, and medical gas extraction module 11. In an embodiment, the housing 2 comprises medical gas container 4, pressure regulator module 5, flow control module 6, pressure valves 7, flow sensor 8, medical gas delivery connection module 9, medical gas extraction connection module 12, medical gas sensor 13, medical gas pump 14, flow sensor 15, safety absorption module 16, and electronic controller 17. As depicted in the figure, pressure regulator module 5, flow control module 6, pressure valves 7, flow sensor 8, and medical gas delivery connection module 9 form medical gas delivery circuit A, while medical gas extraction connection module 12, medical gas sensor 13, medical gas pump 14, and flow sensor 15 form medical gas extraction circuit B. Further, medical gas delivery circuit A and medical gas extraction circuit B combine to form the medical gas delivery system 3.

The housing 2 securely holds the system components for efficient and safe administration of the medical gas, e.g., UNO. In embodiments, the housing 2 can be individualized to each patient's treatment. In embodiments, the housing 2 allows for ease of use, repacking upon system return, and a safety mechanism for handling excess medical gas.

The medical gas delivery system 3 comprises the electromechanical components that modulate the pressure and flow rate from the medical gas container 4 to the target tumor 50 and includes associated safety control systems.

The medical gas container 4 contains the medical gas, which is delivered to the tissue 50 via the medical gas delivery circuit A and the medical gas delivery module 9.

The medical gas container 4 contains the medical gas and typically comprises an outlet regulator, manifold and associated valves mounted thereon and connected to the delivery circuit A for further delivery to the tissue 50. The medical gas container 4 is optionally and preferably reusable.

In various exemplary embodiments of the invention, the volume of the medical gas container 4 is sufficiently small so that the amount of gas in container is not more than the typical gas dose to be delivered to the tissue. According to some embodiments, the medical gas container 4 is of a volume of less than 1 liter, or less than 0.8 liter, or less than 0.75 liter, or less than 0.5 liter, or less than 0.3 liter. In embodiments, the volume of the medical gas container 2 is, but not limited to, less than 100 cc, or less than 90 cc, or less than 80 cc, or less than 70 cc, or less than 60 cc or less than 50 cc. This is particularly advantageous when the gas is toxic, as in the case of gNO at levels above 22,000 ppm, because in the event of undesired leakage of the gas into the treating room, the total amount of gas that can be leaked is small, compared to the size of the room, thus reducing the risk of inhaling a hazardous concentration of the gas by the subject or medical personnel.

For example, when the gas is gNO, the immediately dangerous to life or health (IDLH) concentration is 100 ppm, and so the amount of gNO in container 4 is preferably less than 1/10000 of a typical volume of a treating room, which is typically from about 40,000 liters to about 60,000 liters. Thus, the volume of container 4 can be from about 1 to 10 liters, and it can be filled with the gas at a volumetric concentration of from thousand ppm to several hundred-thousands ppm (e.g., 1,000-1,000,000 ppm), where “ppm” (parts per million) refers to the fraction (e.g., volumetric fraction) of the gas in a gas carrier. The gas carrier can be an inert gas such as nitrogen or argon, preferably nitrogen.

The gas pressure in medical gas container 4 is preferably low, e.g., less than 5 bar, e.g., from about 1 bar to about 5 bar. Alternatively, the gas pressure in medical gas container 4 can be higher (e.g., from about 5 bar to about 150 bar).

Thus, the medical gas container 4 can comprise from about 1,000 ppm to 1,000,000 ppm of the gas, or any intermediate subrange therebetween, for example, from about 1,000 ppm to about 200,000 ppm, or from about 1,000 ppm to about 100,000 ppm, preferably from about 10,000 ppm to about 500,000 ppm, or from about 10,000 ppm to about 200,000 ppm, or from about 10,000 ppm to about 100,000 ppm, or from about 20,000 ppm to about 100,000 ppm, or from about 25,000 ppm to about 100,000 ppm, or from about 25,000 ppm to about 75,000 ppm, or from about 10,000 ppm to about 50,000 ppm, or from about 50,000 ppm to about 100,000 ppm, including any intermediate values and subranges between any of the foregoing, or is about 50,000 ppm.

Preferably the medical gas is UNO. Preferably, the concentration of UNO is from about 10,000 ppm to about 1,000,000 ppm (1% to 100%), including any intermediate values and subranges therebetween, for example, from about 10,000 ppm to about 200,000 ppm, or from about 10,000 ppm to about 100,000 ppm, or from about 15,000 ppm to about 100,000 ppm, or from about 20,000 ppm to about 100,000 ppm, or from about 25,000 ppm to about 100,000 ppm, or from about 25,000 ppm to about 75,000 ppm, or from about 10,000 ppm to about 50,000 ppm, or from about 50,000 ppm to about 100,000 ppm, including any intermediate values and subranges between any of the foregoing, or is about 50,000 ppm.

Preferably, the concentration of UNO is about 10,000 to 200,000 ppm. Preferably, the concentration of UNO is about 20,000 to 200,000 ppm, preferably about 20,000 to 100,000 ppm.

Preferably, the concentration of UNO is about 200,000 ppm. Preferably, the concentration of UNO is about 100,000 ppm. Preferably, the concentration of UNO is about 50,000 ppm. Preferably, the concentration of UNO is about 25,000 ppm. Preferably, the concentration of UNO is about 20,000 ppm. Preferably, the concentration of UNO is about 10,000 ppm.

Preferably, the concentration of UNO is from about 100,000 ppm to about 200,000 ppm, or from about 200,000 ppm to about 500,000 ppm, or from about 500,000 ppm to about 1,000,000 ppm or from about 50,000 ppm or about 100,000 ppm or about 200,000 ppm or about 500,000 ppm or about 1,000,000 ppm.

The pressure regulator module 5 can comprise a cylinder manifold engagement, initial pressure regulator (e.g., to reduce medical gas cylinder pressure from 20-50 Bar to 3 Bar), a check control valve (e.g., for safety control if inlet pressure is too high), gas filter (e.g., for potential particulate/risk control), and pressure valve with a control orifice (e.g., which can reduce the flow to, for example, from 500 mlpm to 0.5 mlpm).

The flow control module 6 is configured to deliver the gas for a time period of from about 1 second to about 60 minutes at a flow volume of from about 0.00001 LPM to about 1 LPM. In embodiments, the flow control module 6 is configured to deliver a predetermined amount (volume and/or mass) of gas. In embodiments, the flow control module 6 is configured to apply treatment in cycles. For example, the flow control module 6 can pause the delivery after a predetermined amount of time and/or after a predetermined amount (volume and/or mass) of the gas has been delivered, and then, after a predetermined time interval, resume the delivery.

Preferably, the time period can be from about 0.1 second to about 1 hour, or from about 1 second to about 10 minutes, or from about 1 minute to about 10 minutes, or from about 10 seconds to about 10 minutes, or from about 0.1 second to about 10 minutes, or from about 30 seconds to about 3 minutes, or from about 1 minute to about 30 minutes, or from about 10 minutes to about 60 minutes, including any intermediate values and subranges between any of foregoing, or it can be about 30 seconds, about 5 minutes, about 10 minutes, about 30 minutes, or about 60 minutes.

Preferably, the time period that ranges from about 30 seconds to about 30 minutes. Preferably, the time period is about 5 minutes.

In some embodiments of the present invention, the flow control module 6 comprises an orifice controlled by a valve, such as, but not limited to, an on/off valve, which in some embodiments of the present invention can be a solenoid valve.

In some embodiments of the present invention, the flow control module 6 comprises a flow controller and a flow limiter. The gas flows from flow limiter to flow controller via a gas flow line. The flow limiter serves for limiting the flow rate (typically the volumetric flow rate) of the gas before entering flow controller. For example, flow limiter can be an analogue flow controller, equipped with a knob for setting an upper limit on the flow rate of the gas passing through limiter. The flow limiter can in some embodiments of the present invention include an orifice of a diameter selected to limit the maximum flow, thus serving as a flow restrictor. Typically, flow limiters limit the gas flow rate to a value of from about 0.0005 liters per minute (LPM) to about 0.15 LPM, more preferably from about 0.0005 liters per minute (LPM) to about 0.11 LPM. Suitable devices for use as flow limiter include the analogue flow controllers, and the flow restrictors.

Preferably, the gas flow rate is a volumetric flow rate of from about 0.00001 LPM to about 10 LPM, preferably from about 0.00001 LPM and about 1 LPM, preferably from about 0.0001 LPM and about 1 LPM, or from about 0.001 LPM and 0.5 LPM, including any intermediate values and subranges therebetween. For example, the volumetric flow rate can be from about 0.001 LPM to about 0.01 LPM, or from about 0.01 LPM to about 0.1 LPM, or from about 0.1 LPM to about 0.25 LPM, or from about 0.25 LPM to about 0.5 LPM, or from about 0.5 LPM to about 1 LPM, or from about 1 LPM to about 2 LPM, or from about 2 LPM to about 3 LPM, or from about 3 LPM to about 4 LPM, or from about 4 LPM to about 5 LPM, or from about 5 LPM to about 6 LPM, or from about 7 LPM to about 8 LPM, or from about 8 LPM to about 9 LPM, or from about 9 LPM to about 10 LPM, including any intermediate values and subranges therebetween, or it can be, for example, about 0.0001 LPM, or about 0.001 LPM, or about 0.01 LPM, or about 0.1 LPM, or about 1 LPM or about 10 LPM.

Preferably, for a closed circuit system, the gas flow rate is at a volumetric flow rate of from about 0.001 LPM to about 0.5 LPM. Preferably, for a closed circuit system, the gas flow rate is at a volumetric flow rate of about 0.2 LPM.

The pressure valves 7 can comprise a safety shut off valve (e.g., for risk control and to shut of gas from upstream of cylinder during post treatment purge) and a three-port valve (e.g., for pulling in ambient air to during post treatment purge, normal to delivery. The pressure valves 7 can be of any type, such as, but not limited to, a ball valve, a gate valve, a plunger valve, a solenoid, a butterfly valve or the like. In an embodiment, the three-port valve has a first port, a second port, and a third port. The third port is in fluid communication with the medical gas delivery module 10. In various exemplary embodiments of the invention, the fluid communications between the medical gas container 4 and the first port are direct, namely that there are gas delivery lines that respectively connect the medical gas container 4 with the first port, and there are no additional elements that interact with the respective medical gas along these lines. The three-port valve is switchable between a first state in which the first port fluidly connects to the third port, and a second state in which the second port fluidly connects to the third port. During treatment, the three-port valve assumes the first state, and the gas flow from the container 4 to the medical gas delivery module 10 and into the tissue 50. Between treatment sessions, more preferably before and after each treatment session, a purge step is executed by switching the three-port valve to its second state, allowing the purging gas to enter the other components of system 1. The purging can contain one or several pressurizing and depressurizing cycles.

The flow sensor 8 monitors the flow through the medical gas delivery circuit A to the medical gas delivery module 10, and also monitors for any back flow from the medical gas delivery module 9, e.g., in order to initiate a safety shutoff.

The medical gas delivery connection module 9 can be a quick connect device with a check valve. The medical gas delivery connection module 9 is configured to attach the medical gas delivery module 10 to the housing 2, with the check valve forming a gas tight seal for the medical gas delivery module 10.

The medical gas delivery module 10 can comprise a male adaptor with check valve, a delivery tubing, and a delivery applicator. The male adaptor with check valve connects the delivery tubing to the medical gas delivery circuit A and also allows the delivery tubing to be gas tight. The delivery tubing communicates medical gas from the medical gas delivery circuit A to the delivery applicator. The delivery applicator can be of any type that has an outlet through which a flow of gas can exit. Typically, but not necessarily, the delivery applicator is a transcutaneous device, e.g., a perforated catheter or a needle depending on the physiological area or treatment of the respective tumor. The different delivery applicators for consideration are the following, but not limited to: perforated spray needle, non-perforated and non-spray needle, umbrella needle, hypodermic needles, transcutaneous or intravenous catheters, micro catheters, microneedles, cannulas, and/or transdermal nitric donors. The needle can optionally be nano size, micron size or macro size needles.

The medical gas extraction module 11 can comprise an extraction membrane (e.g., used to attach delivery applicator into exhaust tubing for a gas tight fit), extraction tubing (e.g., for removal of the medical gas), one-way check valve (e.g., to pull in ambient air for dilution), extraction manual pump (e.g., for risk control if automated pump was to fail, so line could be cleared of the medical gas), a biofilter (e.g., for risk control to protect the delivery system from biological contamination coming from the delivery applicator/patient), and bespoke male adaptor with check valve (e.g., in order to connect the make medical gas extraction module 11 to the housing 2 and make extraction tubing gas tight).

The medical gas extraction connection module 12 can be a quick connect device with a check valve. The medical gas extraction connection module 8 is configured to attach the medical gas extraction module 11 to the housing 2, with the check valve forming a gas tight seal for the medical gas extraction module 11.

The medical gas sensor 13 is configured to detect the presence of any medical gas in the extraction tubing.

The medical gas pump 14 is a pump (e.g., 10 lpm or more) that is configured to pull medical gas and ambient air from the extraction tubing (e.g., to cause dilution in the extraction tubing as to avoid the medical gas being anywhere but in the delivery side of the system).

The flow sensor 15 monitors the flow through the medical gas extraction circuit B in order to determine pump is working and diluting and can also initiate a safety shutoff.

The safety absorption module 16 enables the neutralization of the medical gas.

The electronic controller 17 controls and monitors the system components to ensure no fault states (e.g., for safety control).

In embodiments of the present invention, the medical gas extraction circuit Bis in communication with the delivery applicator through the extraction tubing, which is in fluid communication with the distal end of the delivery applicator, to withdraw excess medical gas (i.e., medical gas that does not enter the tissue 50 from the delivery applicator) exiting the distal end of the delivery applicator.

In embodiments, the distal end of the extraction tubing comprises the gas impermeable extraction membrane. The distal end of the delivery applicator is applied through the gas impermeable extraction membrane of the extraction tubing, thereby creating an airtight seal. In use, excess medical gas that does not enter tissue 50 flows into the extraction tubing instead of being released to the environment. The withdrawn excess medical gas that enters the extraction tubing is returned to the system through the medical gas extraction circuit B. Once the withdrawn excess gas has passed through the medical gas extraction circuit B, it flows through the safety absorption module 14 selected to remove hazardous medical gas components such as gNO and NO₂. For example, safety absorption module 14 can be a potassium permanganate, sodium hydroxide solution, metal oxides, activated charcoal, sodalime, or alkaline activated carbon filter. Safety absorption module 14 can be installed in the system, as illustrated in FIG. 1, or alternatively at a location along the gas flow line after the medical gas extraction circuit B. Once through the safety absorption module 14, the withdrawn excess medical gas, which has now been cleaned of hazardous medical gas components, such as gNO and NO₂, can be evacuated to an exhaust pipe, e.g., a medical center pipe (not shown). In addition, the safety absorption module 14 can also be used absorb any unintentional leaks from the medical gas container 4. In this regard, the safety absorption module 14 is configured to absorb the total volume of the medical gas container 4.

In some embodiments of the present invention, the electronic controller 17 has a circuit configured to automatically control the flow control module 6. For clarity of presentation, control lines from and to the electronic controller 17 are not illustrated.

The electronic controller 17 is configured to control the flow control module 6 according to a predetermined gas flow rate, and/or a predetermined total amount of the gas flowing through the system, and/or a predetermined total amount of time in which the gas flows through the system. The electronic controller 17 can comprise a dedicated circuitry and/or a general purpose computer, configured for receiving data and executing the operations described below. The electronic controller 17 can also include a user interface, e.g., a graphical user interface, for receiving input from the operator. For example, the electronic controller 17 can receive via user interface an input flow rate and/or desired duration for the gas, and automatically select the tuning coefficients.

The electronic controller 17 can also receive via user interface an input dose of the gas to be delivered to the subject and transmits a control signal to the flow control module 6 to ensure that the total amount of delivered gas does not exceed the input dose. For example, the electronic controller 17 can receive from the digital or analog mass flow controller a monitoring signal pertaining to the amount of gas that exits outlet and transmit a stop signal to the system once the amount of gas has reached the dose. In some optional embodiments of the invention the electronic controller 17 also controls one or more of valves, to ensure that the amount of gas delivered does not exceed the input dose.

In some embodiments of the present invention, the user interface can also display certain monitored parameters, e.g., dose, flow rate, duration, pressure.

In some embodiments of the present invention, the electronic controller 17 is configured to also control medical gas delivery circuit A. In some embodiments of the present invention, the electronic controller 17 is configured to also control medical gas extraction circuit B. In these embodiments the electronic controller 17 activates the system at the beginning of the treatment session and deactivates it after the end of the treatment session.

In some embodiments of the present invention, the system 1 comprises an adjustable pressure regulator, in fluid communication with the third port of the pressure valves 6. The gas flows from the third port to the regulator via a gas flow line. The pressure regulator is preferably configured for maintaining a pressure which is below a predetermined threshold when the pressure valves 6 assume their first state (treatment session), and a pressure which is above the predetermined threshold when the pressure valves 6 assume their second state (between treatment sessions). A typical pressure threshold employed by the pressure regulator is, without limitation, from about 2 bars to about 5 bars. The pressure regulator typically includes one or more pressure gauge devices for providing indication regarding the gas pressure downstream and/or upstream the regulator.

In an embodiment, the system 1 would be able to maintain a delivery in a way that provides a constant, high concentration of gaseous nitric oxide (UNO), for a period of time as described herein, at constant or intermittent flow rate as described herein.

In embodiments, the invention provides an “open circuit” system configuration, wherein the open circuit system configuration is identical to the “closed circuit” system configuration except that there is no medical gas extraction module in communication with the delivery applicator to return excess medical gas to the medical gas extraction circuit.

In the open circuit system, the concentration of the medical gas and the time period for the delivery of the medical gas can be the same as in the closed circuit system; however, the open circuit system operates at a lower gas flow rate.

FIG. 1B illustrates one embodiment of an “open circuit” system configuration according to the present invention, wherein the “open circuit” system configuration is similar to the “closed circuit” system configuration except the open circuit system does not include the medical gas extraction module 11 and, therefore, no excess medical gas is returned to the system. All other elements of this open circuit system are the same as described above for the closed circuit system. In some embodiment of the present invention, the user interface can be used to select between the “closed circuit” system configuration and the “open circuit” system configuration. In this regard, the electronic controller 17 can receive via user interface a desired system configuration, and then automatically select a valve configuration in the flow control module 6 corresponding to the desired system configuration.

FIG. 1C illustrates another embodiment of an “open circuit” system configuration according to the present invention, wherein the “open circuit” system configuration is similar to the “closed circuit” system configuration except the open circuit system does not include the medical gas extraction circuit B or the medical gas extraction module 11 and, therefore, no excess gas is returned to the system.

As depicted in FIG. 1C, system 20 comprises a housing 21 and medical gas delivery module 30. In an embodiment, the housing 21 comprises medical gas container 22, pressure regulator module 23, flow control module 24, pressure valves 25, flow sensor 26, medical gas delivery connection module 27, safety absorption module 28, and electronic controller 29. The pressure regulator module 23, flow control module 24, pressure valves 25, flow sensor 26, and medical gas delivery connection module 27 form medical gas delivery circuit C.

The housing 21 securely holds the system components for efficient and safe administration of the medical gas, e.g., UNO. In embodiments, the housing 21 can be individualized to each patient's treatment. In embodiments, the housing 21 allows for ease of use, repacking upon system return, and a safety mechanism for handling excess medical gas.

The housing 21 comprises the electromechanical components that modulate the pressure and flow rate from the medical gas container 22 to the target tumor 150 and includes associated safety control systems.

The medical gas container 22 contains the medical gas, which is delivered to the tissue 150 via the medical gas delivery circuit C and the medical gas delivery module 30.

The medical gas container 22 contains the medical gas and typically comprises an outlet regulator, manifold and associated valves mounted thereon and connected to the delivery circuit C for further delivery to the tissue 150. The medical gas container 22 is optionally and preferably reusable.

In various exemplary embodiments of the invention, the volume of the medical gas container 22 is sufficiently small so that the amount of gas in container is not more than the typical gas dose to be delivered to the tissue. According to some embodiments, the medical gas container 22 is of a volume of less than 1 liter, or less than 0.8 liter, or less than 0.75 liter, or less than 0.5 liter, or less than 0.3 liter. In embodiments, the volume of the medical gas container 22 is, but not limited to, less than 100 cc, or less than 90 cc, or less than 80 cc, or less than 70 cc, or less than 60 cc or less than 50 cc. This is particularly advantageous when the gas is toxic, as in the case of gNO at levels above 22,000 ppm, because in the event of undesired leakage of the gas into the treating room, the total amount of gas that can be leaked is small, compared to the size of the room, thus reducing the risk of inhaling a hazardous concentration of the gas by the subject or medical personnel.

For example, when the gas is gNO, the immediately dangerous to life or health (IDLH) concentration is 100 ppm, and so the amount of gNO in container 22 is preferably less than 1/10000 of a typical volume of a treating room, which is typically from about 40,000 liters to about 60,000 liters. Thus, the volume of container 22 can be from about 1 to 10 liters, and it can be filled with the gas at a volumetric concentration of from thousand ppm to several hundred-thousands ppm (e.g., 1,000-1,000,000 ppm). The gas carrier can be an inert gas such as nitrogen or argon, preferably nitrogen.

The gas pressure in medical gas container 22 is preferably low, e.g., less than 5 bar, e.g., from about 1 bar to about 5 bar. Alternatively, the gas pressure in medical gas container 22 can be higher (e.g., from about 5 bar to about 150 bar).

Thus, the medical gas container 22 can comprise from about 1,000 ppm to 1,000,000 ppm of the gas, or any intermediate subrange therebetween, for example, from about 1,000 ppm to about 200,000 ppm, or from about 1,000 ppm to about 100,000 ppm, preferably from about 10,000 ppm to about 500,000 ppm, or from about 10,000 ppm to about 200,000 ppm, or from about 10,000 ppm to about 100,000 ppm, or from about 20,000 ppm to about 100,000 ppm, or from about 25,000 ppm to about 100,000 ppm, or from about 25,000 ppm to about 75,000 ppm, or from about 10,000 ppm to about 50,000 ppm, or from about 50,000 ppm to about 100,000 ppm, including any intermediate values and subranges between any of the foregoing, or is about 50,000 ppm.

Preferably the medical gas is UNO. Preferably, the concentration of UNO is from about 10,000 ppm to about 1,000,000 ppm (1% to 100%), including any intermediate values and subranges therebetween, for example, from about 10,000 ppm to about 200,000 ppm, or from about 10,000 ppm to about 100,000 ppm, or from about 15,000 ppm to about 100,000 ppm, or from about 20,000 ppm to about 100,000 ppm, or from about 25,000 ppm to about 100,000 ppm, or from about 25,000 ppm to about 75,000 ppm, or from about 10,000 ppm to about 50,000 ppm, or from about 50,000 ppm to about 100,000 ppm, including any intermediate values and subranges between any of the foregoing, or is about 50,000 ppm.

Preferably, the concentration of UNO is about 10,000 to 200,000 ppm. Preferably, the concentration of UNO is about 20,000 to 200,000 ppm, preferably about 20,000 to 100,000 ppm. Preferably, the concentration of UNO is about 200,000 ppm. Preferably, the concentration of UNO is about 100,000 ppm. Preferably, the concentration of UNO is about 50,000 ppm. Preferably, the concentration of UNO is about 25,000 ppm. Preferably, the concentration of UNO is about 20,000 ppm. Preferably, the concentration of UNO is about 10,000 ppm.

Preferably, the concentration of UNO is from about 100,000 ppm to about 200,000 ppm, or from about 200,000 ppm to about 500,000 ppm, or from about 500,000 ppm to about 1,000,000 ppm or from about 50,000 ppm or about 100,000 ppm or about 200,000 ppm or about 500,000 ppm or about 1,000,000 ppm.

The pressure regulator module 23 can comprise a cylinder manifold engagement, initial pressure regulator (e.g., to reduce medical gas cylinder pressure from 20-50 Bar to 3 Bar), a check control valve (e.g., for safety control if inlet pressure is too high), gas filter (e.g., for potential particulate/risk control), and pressure valve with a control orifice (e.g., which can reduce the flow to, for example, from 500 mlpm to 0.5 mlpm).

The flow control module 24 is configured to deliver the gas for a time period of from about 1 second to about 60 minutes at a flow volume of from about 0.00001 LPM to about 1 LPM. In embodiments, the flow control module 24 is configured to deliver a predetermined amount (volume and/or mass) of gas. In embodiments, the flow control module 24 is configured to apply treatment in cycles. For example, the flow control module 24 can pause the delivery after a predetermined amount of time and/or after a predetermined amount (volume and/or mass) of the gas has been delivered, and then, after a predetermined time interval, resume the delivery.

Preferably, the time period can be from about 0.1 second to about 1 hour, or from about 1 second to about 10 minutes, or from about 1 minute to about 10 minutes, or from about 10 seconds to about 10 minutes, or from about 0.1 second to about 10 minutes, or from about 30 seconds to about 3 minutes, or from about 1 minute to about 30 minutes, or from about 10 minutes to about 60 minutes, including any intermediate values and subranges between any of foregoing, or it can be about 30 seconds, about 5 minutes, about 10 minutes, about 30 minutes, or about 60 minutes.

Preferably, the time period that ranges from about 30 seconds to about 30 minutes. Preferably, the time period is about 5 minutes.

In some embodiments of the present invention, the flow control module 6 comprises an orifice controlled by a valve, such as, but not limited to, an on/off valve, which in some embodiments of the present invention can be a solenoid valve.

In some embodiments of the present invention, the flow control module 6 comprises a flow controller and a flow limiter. The gas flows from flow limiter to flow controller via a gas flow line. The flow limiter serves for limiting the flow rate (typically the volumetric flow rate) of the gas before entering flow controller. For example, flow limiter can be an analogue flow controller, equipped with a knob for setting an upper limit on the flow rate of the gas passing through limiter. The flow limiter can in some embodiments of the present invention include an orifice of a diameter selected to limit the maximum flow, thus serving as a flow restrictor. Typically, flow limiters limit the gas flow rate to a value of from about 0.0005 liters per minute (LPM) to about 0.15 LPM, more preferably from about 0.0005 liters per minute (LPM) to about 0.11 LPM. Suitable devices for use as flow limiter include the analogue flow controllers, and the flow restrictors.

Preferably, the gas flow rate is a volumetric flow rate of from about 0.00001 LPM to about 10 LPM, preferably from about 0.00001 LPM and about 1 LPM, preferably from about 0.0001 LPM and about 1 LPM, or from about 0.001 LPM and 0.5 LPM, including any intermediate values and subranges therebetween. For example, the volumetric flow rate can be from about 0.001 LPM to about 0.01 LPM, or from about 0.01 LPM to about 0.1 LPM, or from about 0.1 LPM to about 0.25 LPM, or from about 0.25 LPM to about 0.5 LPM, or from about 0.5 LPM to about 1 LPM, or from about 1 LPM to about 2 LPM, or from about 2 LPM to about 3 LPM, or from about 3 LPM to about 4 LPM, or from about 4 LPM to about 5 LPM, or from about 5 LPM to about 6 LPM, or from about 7 LPM to about 8 LPM, or from about 8 LPM to about 9 LPM, or from about 9 LPM to about 10 LPM, including any intermediate values and subranges therebetween, or it can be, for example, about 0.0001 LPM, or about 0.001 LPM, or about 0.01 LPM, or about 0.1 LPM, or about 1 LPM or about 10 LPM.

Preferably, for an open circuit system, the gas flow rate is at a volumetric flow rate of from about 0.01 mL/min (mLPM) to 10 mLPM (about 0.00001 LPM to about 0.010 LPM). Preferably, for an open circuit system, the gas flow rate is at a volumetric flow rate of about 0.5 mLPM (about 0.0005 LPM).

The pressure valves 25 can comprise a safety shut off valve (e.g., for risk control and to shut of gas from upstream of cylinder during post treatment purge) and a three-port valve (e.g., for pulling in ambient air to during post treatment purge, normal to delivery. The pressure valves 25 can be of any type, such as, but not limited to, a ball valve, a gate valve, a plunger valve, a solenoid, a butterfly valve or the like. In an embodiment, the three-port valve has a first port, a second port, and a third port. The third port is in fluid communication with the medical gas delivery module 30. In various exemplary embodiments of the invention, the fluid communications between the medical gas container 22 and the first port are direct, namely that there are gas delivery lines that respectively connect the medical gas container 22 with the first port, and there are no additional elements that interact with the respective medical gas along these lines. The three-port valve is switchable between a first state in which the first port fluidly connects to the third port, and a second state in which the second port fluidly connects to the third port. During treatment, the three-port valve assumes the first state, and the gas flow from the container 22 to the medical gas delivery module 30 and into the tissue 150. Between treatment sessions, more preferably before and after each treatment session, a purge step is executed by switching the three-port valve to its second state, allowing the purging gas to enter the other components of system 20. The purging can contain one or several pressurizing and depressurizing cycles.

The flow sensor 26 monitors the flow through the medical gas delivery circuit C to the medical gas delivery module 30, and also monitors for any back flow from the medical gas delivery module 30, e.g., in order to initiate a safety shutoff.

The medical gas delivery connection module 27 can be a quick connect device with a check valve. The medical gas delivery connection module 27 is configured to attach the medical gas delivery module 30 to the housing 21, with the check valve forming a gas tight seal for the medical gas delivery module 30.

The medical gas delivery module 30 can comprise a male adaptor with check valve, a delivery tubing, and a delivery applicator. The male adaptor with check valve connects the delivery tubing to the medical gas delivery circuit C and also allows the delivery tubing to be gas tight. The delivery tubing communicates medical gas from the medical gas delivery circuit C to the delivery applicator. The delivery applicator can be of any type that has an outlet through which a flow of gas can exit. Typically, but not necessarily, the delivery applicator is a transcutaneous device, e.g., a perforated catheter or a needle depending on the physiological area or treatment of the respective tumor. The different delivery applicators for consideration are the following, but not limited to: perforated spray needle, non-perforated and non-spray needle, umbrella needle, hypodermic needles, transcutaneous or intravenous catheters, micro catheters, microneedles, cannulas, and/or transdermal nitric donors. The needle can optionally be nano size, micron size or macro size needles.

The safety absorption module 28 enables the neutralization of the medical gas during, e.g., any unintentional leaks from the medical gas container 4. In this regard, the safety absorption module 28 is configured to absorb the total volume of the medical gas container 4.

The electronic controller 29 controls and monitors the system components to ensure no fault states (e.g., for safety control).

In some embodiments of the present invention, the electronic controller 29 has a circuit configured to automatically control the flow control module 24. For clarity of presentation, control lines from and to the electronic controller 29 are not illustrated.

The electronic controller 29 is configured to control the flow control module 24 according to a predetermined gas flow rate, and/or a predetermined total amount of the gas flowing through the system, and/or a predetermined total amount of time in which the gas flows through the system. The electronic controller 29 can comprise a dedicated circuitry and/or a general purpose computer, configured for receiving data and executing the operations described below. The electronic controller 29 can also include a user interface, e.g., a graphical user interface, for receiving input from the operator. For example, the electronic controller 29 can receive via user interface an input flow rate and/or desired duration for the gas, and automatically select the tuning coefficients.

The electronic controller 29 can also receive via user interface an input dose of the gas to be delivered to the subject and transmits a control signal to the flow control module 24 to ensure that the total amount of delivered gas does not exceed the input dose. For example, the electronic controller 29 can receive from the digital or analog mass flow controller a monitoring signal pertaining to the amount of gas that exits outlet and transmit a stop signal to the system once the amount of gas has reached the dose. In some optional embodiments of the invention the electronic controller 29 also controls one or more of valves, to ensure that the amount of gas delivered does not exceed the input dose.

In some embodiments of the present invention, the user interface can also display certain monitored parameters, e.g., dose, flow rate, duration, pressure.

In some embodiments of the present invention, the electronic controller 29 is configured to also control medical gas delivery circuit C. In these embodiments, the electronic controller 29 activates the system at the beginning of the treatment session and deactivates it after the end of the treatment session.

In some embodiments of the present invention, the system 20 comprises an adjustable pressure regulator, in fluid communication with the third port of the pressure valves 25. The gas flows from the third port to the regulator via a gas flow line. The pressure regulator is preferably configured for maintaining a pressure which is below a predetermined threshold when the pressure valves 25 assume their first state (treatment session), and a pressure which is above the predetermined threshold when the pressure valves 25 assume their second state (between treatment sessions). A typical pressure threshold employed by the pressure regulator is, without limitation, from about 2 bars to about 5 bars. The pressure regulator typically includes one or more pressure gauge devices for providing indication regarding the gas pressure downstream and/or upstream the regulator.

In an embodiment, the system 20 would be able to maintain a delivery in a way that provides a constant, high concentration of gaseous nitric oxide (UNO), for a period as described herein, and at constant or intermittent gas flow rate as described herein.

The invention also provides a method for delivering a medical gas to a tissue in a subject in need thereof using a closed-circuit system or an open-circuit system as described herein. Preferably, the medical gas is delivered to the tissue using a closed-circuit system as described herein. Preferably the medical gas is delivered to the tissue using a open-circuit system as described herein. Preferably, the tissue is a cancerous tissue. Preferably, the tissue is a tumor.

The closed-circuit system or an open-circuit system as described herein can be used to treat cancer in a subject in need thereof.

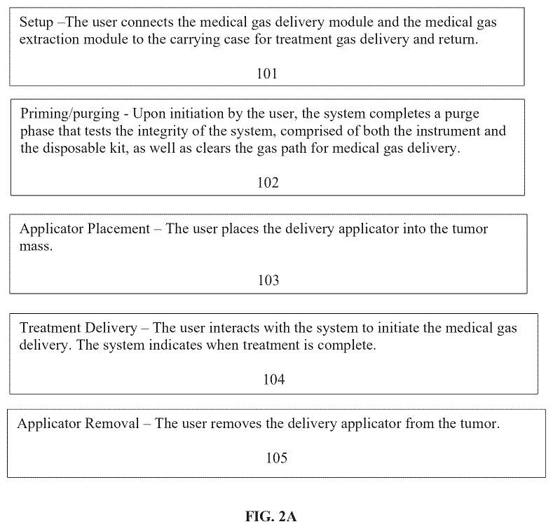

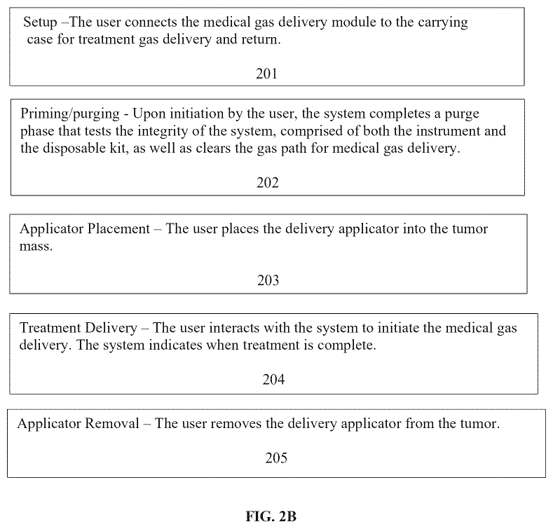

FIG. 2A illustrates an exemplary method suitable for delivery of a medical gas to a tissue using the “closed circuit” system as described herein. The method comprises the following steps: setup, priming/purging, applicator placement, treatment delivery, and applicator removal. As depicted in the figure, during setup, e.g., step 101, a user can connect at least one of the medical gas delivery module 9 and the medical gas extraction module 11 to the housing 2 for medical gas delivery and return. Then, during priming/purging, e.g., step 102, the user initiates the system 1 to complete a purge phase that tests the integrity of the system 1, comprised of both the instrument and the disposable kit, as well as clears the gas path for medical gas delivery. Then, during applicator placement, e.g., step 103, the user can place the delivery applicator into the tumor mass 10. Then, during treatment delivery, e.g., step 104, the user can interact with the system 1 to initiate the medical gas delivery, wherein the system 1 will indicate when the treatment is complete. Then, during applicator removal, e.g., step 105, the user can remove the delivery applicator from the tumor.

FIG. 2B illustrates an exemplary method suitable for delivery of a gas to a tissue using the “open circuit” system as described herein. The method comprises the following steps: setup, priming/purging, applicator placement, treatment delivery, and applicator removal. As depicted in the figure, during setup, e.g., step 201, a user can connect the medical gas delivery module 30 to the housing 21 for medical gas delivery and return. Then, during priming/purging, e.g., step 202, the user initiates the system 20 to complete a purge phase that tests the integrity of the system 20, comprised of both the instrument and the disposable kit, as well as clears the gas path for medical gas delivery. Then, during applicator placement, e.g., step 203, the user can place the delivery applicator into the tumor mass 30. Then, during treatment delivery, e.g., step 204, the user can interact with the system 20 to initiate the medical gas delivery, wherein the system 20 will indicate when the treatment is complete. Then, during applicator removal, e.g., step 205, the user can remove the delivery applicator from the tumor.

In any embodiment herein, when the medical gas is nitric oxide, the gNO is preferably of medical purity, that is, preferably at least about 95 %, more preferably at least about 99 %, and even more preferably at least about 99.5 % pure gNO. The gNO is preferably provided as a mixture of gNO and other gases, such as air, nitrogen, oxygen, and so forth, preferably an inert gas such as, for example, nitrogen, and its ppm concentration is within the gas it is mixed with.

It is to be noted that a certain level of damage to collateral cells may be tolerated, and that the conditions under which the gNO gas is administered may be optimized to decrease damage to collateral cells while also providing the therapeutic effects described herein.

According to any of the embodiments, the medical gas is administered at a concentration as described above, for a time period as described above, and at a gas flow rate as described above. Preferably, the medical gas is gNO, preferably UNO.

Where a closed circuit system is being used to deliver the medical gas to a tissue, the closed circuit system can operate at a higher gas flow rate (compared to the open circuit system) as any medical gas that is not absorbed by or into the tissue (i.e., excess medical gas) is extracted through the medical gas extraction module and returned to the medical gas extraction circuit for scavenging. In contrast, where an open circuit system is being used to deliver the medical gas to a tissue, the open circuit system operates a low gas flow rate (compared to the closed circuit system) to provide the medical gas at a rate comparable to the rate at which the tissue is absorbing the medical gas in order to minimize any excess medical gas from leaving the tissue and entering the ambient air and/or prevent swelling of the tissue. Any excess medical gas that is not absorbed by the tissue and enters the ambient air, if any, is being delivered at a low volumetric gas flow rate that the total amount of gas that can be leaked is small, compared to the size of the room, thus reducing the risk of inhaling a hazardous concentration of the gas by the subject or medical personnel.

According to any of the embodiments described herein, the gNO is administered, as described herein in any of the respective embodiments, in an amount of no more 1 mg gNO per 100 mm³ tumor volume, per administration, so as to avoid possible damage to healthy tissues adjacent to, or surrounding, the treated tumor.

According to any of the embodiments described herein, the gNO is administered, as described herein in any of the respective embodiments, in an amount of about 250 mg per cm³ tumor, per administration.

According to any of the embodiments described herein, the gNO is administered, as described herein in any of the respective embodiments, in an amount of from about 0.01 mg to about 100 mg, or from about 0.1 to about 10 mg per a tumor of 20 mm 3 or less, including any intermediate values and subranges therebetween.

According to any of the embodiments described herein, the gNO is administered, as described herein in any of the respective embodiments, in an amount of from about 0.1 to about 300 mg including any intermediate values and subranges therebetween, per 1 cm³ tumor, per administration. For example, the gNO is administered in an amount of from about 0.1 mg to about 250 mg, or from 0.1 mg to about 100 mg, or from 1 mg to about 50 mg, or from about 1 mg to about 100 mg, or from about 1 mg to about 300 mg, of from about 50 mg to about 100 mg, or from about 50 mg to about 300 mg, or from about 100 mg to about 150 mg, or from about 100 mg to about 300 mg, or from about 10 mg to about 100 mg, or from about 10 mg to about 250 mg, or from about 0.1 mg to about 10 mg, or from about 10 mg to 200 mg, including any intermediate values and subranges of any of the foregoing, per 1 cm³ tumor, per administration.

According to any of the embodiments described herein, the gNO is administered, as described herein in any of the respective embodiments, to a tumor having a volume of up to 20 mm³, and an amount of gNO that is administered as described herein in any of the respective embodiments is from about 0.001 mg to about 10 mg, or from about 0.01 mg to about 20 mg, or from about 0.01 mg to about 2 mg, or from about 0.1 mg to about 1.0 mg, or from about 0.2 mg to about 0.8 mg, including any intermediate values and subranges between any of the foregoing, per administration.

Prior to delivering the medical gas to the tissue, the system can be primed by a flow of purging gas through the system. Priming tests the integrity of the system as well as clearing the gas path for medical gas delivery.

After treatment, the system can then be purged with a purging gas to remove any excess medical gas in the system and scavenge the excess medical gas, if applicable (e.g., in the closed system).

Without being bound by any particular theory, it is assumed that a high dose (concentration or amount) as described herein in any of the respective embodiments, inhibits the growth of tumor cells, reduces tumor volume and/or stimulates an anti-tumor immune response, as described herein in any of the respective embodiments, without causing a harmful effect to healthy tissues in the vicinity of the tumor.

For any of the embodiments described herein for administration of gNO, the administration can be either continuous or pulsed (e.g., intermittent), such that for each administration, the indicated dose of gNO is administered either continuously or in a pulsed manner. When the dose is referred to in ppm units, each pulse is at the indicated dose concentration, as described herein in any of the respective embodiments. When the dose is referred to as the total mass per administration, the indicated dose is divided into pulses.

According to any of the embodiments described herein, gNO is pulsed from about 2 to about 50 times, or from about 2 to about 30 times, or from about 2 to about 20 times, or from about 2 to about 15 times, or from about 5 to about 15 times, including any intermediate values and subranges therebetween, or about 10 times, per administration.

According to any of the embodiments described herein, each pulse is between about 1,000 ppm and about 1,000,000 ppm, or between 4000 ppm and about 1,000,000 ppm gNO, or between 10,000 ppm and about 1,000,000 ppm gNO, at a volumetric flow (flow volume) of from about 0.00001 LPM to about 0.5 LPM, wherein each pulse is, independently, between about 0.1 second and about 10 minutes per pulse with a break of from about 0.1 second to about 10 minutes between pulses.

According to any of the embodiments described herein, each pulse of gNO is, independently, from about 10 seconds per pulse to about 45 seconds per pulse, including any intermediate values and subranges therebetween.

According to any of the embodiments described herein, each pulse of gNO is about 30 seconds per pulse.

According to any of the embodiments described herein, gNO is not administered between pulses and the time between each two pulses is, independently, from about 1 second to about 300 seconds, or from about 1 second to about 200 seconds, or from about 1 second to about 100 seconds, or from about 1 second to about 50 seconds, or from about 10 seconds to about 50 seconds, including any intermediate values and subranges therebetween, or is about 20 seconds.

According to any of these embodiments, the ratio between the time of gNO pulsed administration and the resting time between pulses ranges from 1:2 to 1:5. For example, for each pulse of gNO administration during 5 seconds, a following resting time is independently from 10 to 50 seconds. Preferably, the gNO is pulsed such that about 33 % of the time gNO is delivered and 66 % of the time is resting or waiting time between pulses.

According to any of the embodiments described herein, the gNO is administered, as described herein in any of the respective embodiments, at two or more administration sites in or on the tumor (depending in the administration mode). In some of these embodiments, the distance between the two administration sites is, independently, from about 2.5 mm to about 1 cm, or from about 0.25 cm to about 0.5 cm, including any intermediate values and subranges therebetween.

When gNO is administered to two or more tumor sites, each administration is at the ppm dose or mass amount indicated herein in any of the respective embodiments, or the total mass (amount) administered to all tumor sites is as indicated herein in any of the respective embodiments.

According to any of the embodiments described herein for high dose administration of gNO, the administration is performed one or more times per treatment session.

In some embodiments, it is performed once during a treatment session. In some embodiments, it is performed twice, thrice, or more times during a treatment session. In some of these embodiments, the administration is performed once daily during the treatment session. In some embodiments, multiple treatment sessions with allowable time between treatments are performed. Preferably, the administration is performed such that a time interval between the two administrations is at least one week and in other instances up to 6 weeks, during a treatment session.

The duration of a treatment session can be determined by skilled persons such as physicians, in accordance with the subject's response to the treatment, that is, in accordance with the effect of the treatment on the growth of the cells of the primary and/or secondary tumor, as described herein. According to any of the embodiments described herein for administration of gNO, the administration is performed once a day, although two or more times a day are also contemplated.

As used herein the term “about” refers to ±10 %.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention.

Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

While this invention has been particularly shown and described with references to preferred embodiments thereof, it will be understood by those skilled in the art that various changes in form and details may be made therein without departing from the scope of the invention encompassed by the appended claims.

1. A system for delivering medical gas to a tissue, the system comprising: a housing containing:
a medical gas container including a medical gas, a medical gas delivery circuit, a medical gas extraction circuit, and a safety absorption module;
a medical gas delivery module; and
a medical gas extraction module, wherein the medical gas delivery module is attached to the medical gas delivery circuit to allow the medical gas to be delivered to the tissue, and the medical gas extraction module is in communication with the medical gas delivery module to return excess medical gas to the medical gas extraction circuit.

2. The system of claim 1, wherein the medical gas is gaseous nitric oxide (gNO).

3. The system of claim 1, wherein the medical gas container comprises 1,000 ppm to 1,000,000 ppm of gNO.

4. The system of claim 1, wherein a total volume of the medical gas in the medical gas container is delivered to the tissue.

5. The system of claim 1, wherein the medical gas delivery circuit comprises one or more valves, one or more regulators, one or more cylinder manifolds, and one or more flow sensors.

6. The system of claim 5, wherein the medical gas delivery circuit comprises a gas manifold, a pressure regulator, a first valve, a flow sensor, a gas filter, and a second valve, wherein the flow sensor is arranged after the first and second valves.

7. The system of claim 6, wherein the second valve is a three-port valve having a first port for receiving the medical gas from the medical gas container, a second port for receiving a purging gas, and a third port in communication with the delivery tubing.

8. The system of claim 1, wherein the medical gas delivery circuit further comprises one of a flow metering and a flow control system.

9. The system of claim 8, wherein the flow control system is at least one of a flow limiter and flow controller.

10. The system of claim 9, wherein the flow controller is a mass flow controller.

11. The system of claim 8, wherein the flow control system is configured to deliver the medical gas for a time period of from about 1 second to about 60 minutes at a flow volume of from about 0.00001 LPM to about 1 LPM.

12. The system of claim 1, wherein the medical gas extraction circuit comprises a connection port for the extraction tubing, a gas sensor, a pump, and a flow sensor.

13. The system of claim 1, wherein the safety absorption module is in communication with at least one of (i) the medical gas delivery circuit to neutralize any unwanted leaks of the medical gas during administration and (ii) the medical gas extraction circuit to neutralize any excess medical gas extracted from the tissue.

14. The system of claim 1, wherein the medical gas delivery module comprises medical gas delivery tubing and a delivery applicator.

15. The system of claim 14, wherein the medical gas delivery circuit provides fluid communication between the medical gas container and the delivery tubing and the delivery applicator, wherein the medical gas flows from the medical gas container through the medical gas delivery circuit, the delivery tubing, and the delivery applicator to the tissue.

16. The system of claim 1, wherein the medical gas extraction module comprises medical gas extraction tubing.

17. The system of claim 16, wherein the medical gas delivery tubing is attached to the medical gas delivery circuit to allow the medical gas to be delivered to the tissue through the delivery applicator, and the medical gas extraction tubing is in communication with the delivery applicator to return medical gas to the medical gas extraction circuit.

18. The system of claim 16, wherein the distal end of the medical gas extraction tubing is in communication with the distal end of the delivery applicator, wherein the communication between the delivery applicator and the extraction tubing is airtight.

19.-40. (canceled)
